(19) United States
(12) Patent Application Publication
(10) Pub. No.: US 2004/0243317 A1
(43) Pub. Date: Dec. 2, 2004

(54) Method and computer program product for aligning similarity of two biological sequences

(75) Inventor: Wen-Hsuang Yao, Taipei (TW)

Correspondence Address: NIXON PEABODY, LLP, 401 9TH STREET, NW, SUITE 900, WASHINGTON, DC, 20004-2128, US

(73) Assignee: Industrial Technology Research Institute, Hsinchu (TW)

(21) Appl. No.: 10/609,657

(22) Filed: Jul. 1, 2003

Related U.S. Application Data
(63) Continuation-in-part of application No. 09/741,078, filed on Dec. 21, 2000, now abandoned.

(51) Int. Cl. G06F 19/00; G01N 33/48; G01N 33/50

(52) U.S. Cl. 702/20

(57) ABSTRACT

The present invention provides a method and a computer program product for similarity alignment of two biological sequences, such as nucleotide sequences and amino acid sequences. First of all, a seed pair in two biological sequences is selected by satisfying a user-defined limitation. Two fragments are extended in the same direction. If the data arrangement between the two extended fragments satisfies an extension condition, two other fragments are further extended subsequently. Otherwise, it is then to match the fragments by gap insertion. After gap insertion, if the data arrangement becomes to satisfy the extension condition, then two fragments start to be extended. Otherwise the extension is terminated and resulted fragments are obtained.

This Application, as a continuation in part application, claims priority to U.S. patent application Ser. No. 09/741,078 filed on Dec. 21, 2000.

### FIELD OF INVENTION

The present invention relates to similarity alignment of two biological sequences and also relates to similarity searching for more than two biological sequences in a biological sequence database.

### BACKGROUND OF THE INVENTION

There are many conventional techniques for similarity alignment of biological sequences, such as Gapped-BLAST.

Gapped-BLAST is a kind of heuristically-modified dynamic programming technique. It selects a window length (a distance between two hits) to start extension if other two non-overlapping hits having the same diagonal. However, Gapped-BLAST is an expensive technique because the required computation amount of dynamic programming techniques is proportional to the lengths of two biological sequences to be compared. Therefore, Gapped-BLAST is impractical in searching for similar biological sequences from a large database or matching long biological sequences, such as genome data, without the use of a supercomputer or other special purpose hardware.

Accordingly, there remains a need in the art to provide an improved method with high qualities of both speed and sensitivity when aligning two biological sequences, especially huge genome sequences.

### SUMMARY OF THE INVENTION

The topic stated above is able to be solved by using the alignment method of the present invention. One aspect of the present invention is to provide a method and a computer program product for aligning similarity of two biological sequences. The method includes the steps of: selecting a seed pair of the two biological sequences; respectively extending two fragments adjacent to the seed pair by a predetermined number of successive bases; determining if the extended fragments satisfy an extension condition; if yes, extending respectively two fragments adjacent to the extended fragments by the predetermined number of successive bases and returning to the determining step; if no, respectively selecting two identical sub-fragments from the extended fragments which do not satisfy the extension condition; determining either one of the sub-fragments closer to the seed pair; matching the extended fragments by inserting at least one gap in front of the one of the sub-fragments which is closer to the seed pair; determining if the matched fragments satisfy the extension condition; if yes, respectively extending two fragments adjacent to the matched fragments by the predetermined number of successive bases and returning to the first determining step to determine if the extended fragments satisfy the extension condition; otherwise, stopping extension and obtaining resulted fragments.

The other aspect of the present invention is to search for two similar biological sequences from a database, such as a DNA, protein and polysaccharide databases.

### BRIEF DESCRIPTION OF THE DRAWINGS

FIG. 1 is a flow chart indicating the method of the present invention;

FIG. 2A, FIG. 2B, and FIG. 2C are flow charts showing the steps of obtaining resulted fragments; and

FIG. 3, including FIGS. 3A, 3B, 3C, 3D, and 3E, illustrates the most preferred embodiment of the present invention.

### DETAILED DESCRIPTION

The present invention provides a method and a computer program product to align similarity of two biological sequences, e.g. nucleic acid sequences, protein sequences or polysaccharide sequences. Furthermore, the present invention is able to search for two similar biological sequences from a biological sequence database.

To describe the invention clearly, some term definitions used herein are given as follows.

The term “base” used herein refers to a specific unit in a biological sequence. For example, a base in a nucleic acid sequence means a specific nucleotide, such as A (adenine), T (thymine), C (cytosine) and G (guanine), a base in a protein sequence means a specific amino acid, such as G (Glycine, Gly), A (Alanine, Ala) and L (Leucine, Leu), and a base in a polysaccharide sequence means a monosaccharide, such as Glu (Glucose) and Gal (Galactose).

The term “data” used herein refers to bases forming a biological sequence. For example, data in a nucleic acid sequence mean a nucleotide or nucleotides, data in a protein sequence mean an amino acid or amino acids, and data in a polysaccharide sequence mean a monosaccharide or monosaccharide.

The term “similarity” used herein represents how two biological sequences are similar to each other.

The term “fragment” used herein refers to a part of biological sequence. Taking a DNA sequence with 1000 nucleotides as an example, a fragment of the sequence may be from nucleotide 1 to nucleotide 100. The length of the fragment is defined by users in accordance with different applications or needs.

The term “sub-fragment” used herein means a part of fragment. Taking a fragment including 100 nucleotides as an example, its sub-fragment may be from nucleotide 23 to nucleotide 30. Similarly, users can define a suitable length of a sub-fragment in accordance with their needs.

The term “pattern” of two biological sequences used herein refers to the data type arrangement between the two biological sequences. A pattern can be shown as “cgtaatc”, for example.

The term “condition” used herein means a predefined similarity limitation to determine if two sequences or fragments are similar or not. It is defined by users according to their needs as well. With reference to the definition of “pattern” stated above, when patterns of two fragments satisfy a user-defined condition, it means that the similar percentage between the two fragments is above 50%, for example.

The term “gap” used herein means a space, or a meaningless base, inserted in order to compensate for an originally non-existing base in one sequence to match another sequence. A gap is designated as “-”. For example, 4 successive gaps is shown as “- - - -”.

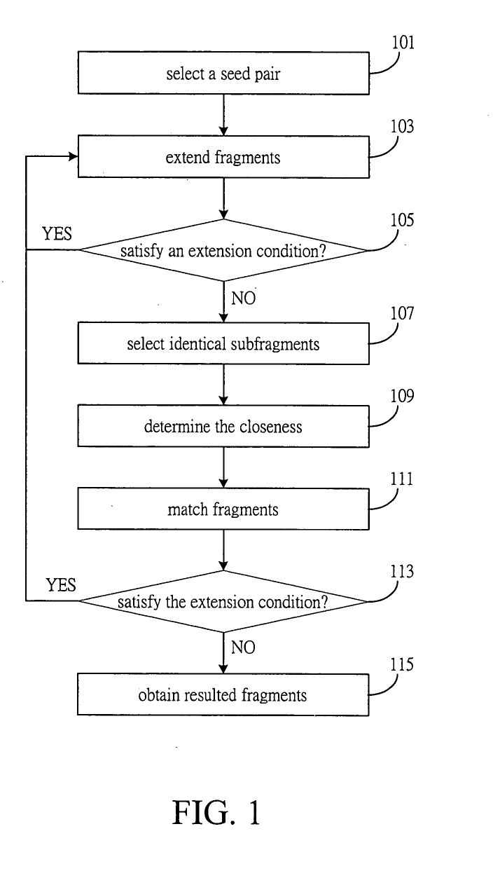

FIG. 1 shows the flow chart of the method provided by the invention. There are two biological sequences to be proceeded. In step 101, a seed pair of the two biological sequences is selected respectively. The method of the present invention does not restrain how to select the seed pair. In other words, one can select the seed pair by any known ways, such as the HSP method of BLAST, or even innovative ways as long as the selected seed pair is able to satisfy a particular limitation according to users' needs. The particular limitation may be having the same length and/or the same data, for example. Once the seed pair has been selected, two other fragments adjacent to the seed pair are extended respectively by a predetermined number of successive bases in the same direction in step 103. It is then determined if the extended fragments satisfy an extension condition in step 105. As set forth hereinbefore, the extension condition is defined according to users' needs. If the extension condition is met, the method returns to step 103 to proceed with the next extension, and two fragments will be extended and adjacent to the extended fragments which have been determined satisfying the extension condition. If the extension condition is not met, then the method continues to step 107 in which two identical sub-fragments in the extended fragments are respectively selected and a base number of the identical sub-fragments is also decided by users. In step 109, both sub-fragments are determined that which one is closer to the seed pair. In step 111, the extended fragments unsatisfying the extension condition are matched by inserting at least one gap in front of the sub-fragment which is closer to the seed pair. The number of required gaps depends on how far the two sub-fragments separate and the inserted gap(s) can make both sub-fragments have corresponding positions. In step 113, it is determined if the matched fragments satisfy the extension condition now. If yes, the method returns to step 103 to extend other fragments adjacent to the matched fragments. Otherwise, the extension process is terminated and resulted fragments are obtained in step 115.

More particularly, in step 103, the number of successive bases for extending two fragments is preferred from 4 to 400. An excessive small number, or less than 4, would make the extension meaningless and inconsiderable results come out. On the contrary, an excessive large number, or larger than 400, would make the extension condition difficult to satisfy in step 105. Therefore, it is suggested that users decide an appropriate number between 4 and 400 based on their needs.

Regarding the extension condition in step 105, it is more efficient to set the extension condition to be having 40%˜100% similarity of base types between the two extended fragments. And it is apparent to those skilled that the higher the similarity is asked to be satisfied, the more matching steps are required to be executed, which means more running time needed.

In step 107, two identical sub-fragments are selected. Theoretically, the base number of the two identical sub-fragments larger than 2 is acceptable to execute the following steps 109 and 111. However, in view of execution speed, it is suggested to set the base number within a range of 3˜400. If one expects to obtain the optimal performance, a range of 3˜50 should be considered.

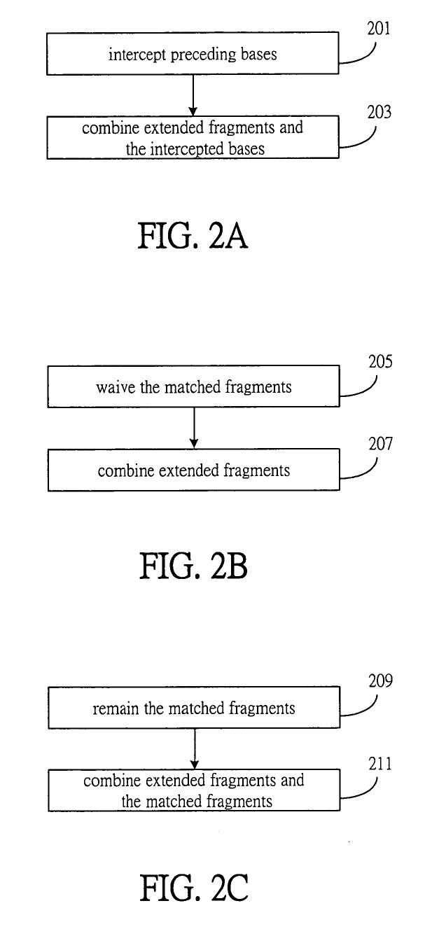

In step 115, the resulted fragments can be obtained by many ways. As FIG. 2A shows, one can intercept and keep preceding substantially identical bases of the matched fragments. For example, if two fragments which do not satisfy the extension condition in step 113 are “gacttagcctgg” and “gact—gcctac”, one might keep “gacttagcct” and “gact—gcct” in step 201, and then combine them and all extended fragments satisfying the extension condition into the resulted fragments in step 203. However, it probably happens that two matched fragments have little similarity on base alignment. Hence, as FIG. 2B shows, the matched fragments are waived in step 205 and only the extended fragments satisfying the extension condition are combined into the resulted fragments in step 207. Alternatively, as FIG. 2C shows, one can remain the whole matched fragments without considering their base similarity in step 209 and directly combine the matched fragments and the extended fragments satisfying the extension condition into the resulted fragments in step 211.

The present invention also provides a computer program product for aligning similarity of two biological sequences. The computer program product comprises a computer readable storage medium which has code segments to execute the aforementioned method. The computer readable storage medium may be a CD-ROM, a floppy disc, a DRAM, a hard drive, a flash media, a tape, or the like.

The code segments at least include a first code segment, a second code segment, a third code segment, a fourth code segment, a fifth code segment, a sixth code segment, and a seventh code segment. The first code segment is configured to select a seed pair of the two biological sequences. The second code segment is configured to respectively extend two fragments by a predetermined number of successive bases, and the two extended fragment are respectively adjacent to the two fragments having extended in the last time. For example, first extended fragments should be next to the seed pair, second extended fragments should be next to the first extended fragments and so on. The third code segment is configured to determine whether the extended fragments satisfy the extension condition. The fourth code segment is configured to respectively select two identical sub-fragments from the extended fragments if the extended fragments are determined not to satisfy the extension condition by the third code segment. The fifth code segment is configured to determine either one of the sub-fragments closer to the seed pair. The sixth code segment is configured to match the extended fragments unsatisfying the extension condition by inserting at least one gap in front of the one of the sub-fragments determined by the fifth code segment. The seventh code segment is configured to obtain the resulted fragments.

The limitations of the predetermined number of successive bases, the extension condition and the base number of the two identical sub-fragments are the same as aforementioned.

After the extension implemented by the second code segment, the extended fragments will be determined by the third code segment. If the pattern satisfies the extension condition, the third code segment, therefore, will send a signal to the second code segment to extend next fragments. Alternatively, the fourth code segment will be activated to select two identical sub-fragments from the extended fragments thereof. After the matching action held by the sixth code segment, the third code segment starts again to determine whether the extension condition is met. If yes, the third code segment will send another signal to the second code segment so that next fragments adjacent to the matched fragments can be extended. Otherwise, the seventh code segment will stop the extension and obtain the resulted fragments.

For the case of intercepting preceding substantially identical bases of the matched fragments while proceeding with the step of obtaining the resulted fragments, the seventh code segment further includes an eighth code segment and a ninth code segment. The eighth code segment is provided to intercept preceding substantially identical bases of the matched fragments. The ninth code segment is provided to combine all of the extended fragments satisfying the extension condition and the intercepted bases generated by the eighth code segment into the resulted fragments.

For the case of waiving the matched fragments while proceeding with the step of obtaining the resulted fragments, the seventh code segment further includes a tenth code segment and an eleventh code segment. The tenth code segment is provided to waive the whole matched fragments and the eleventh code segment is provided to simply combine all of the extended fragments satisfying the extension condition into the resulted fragments.

For the case of remaining the matched fragments while proceeding with the step of obtaining the resulted fragments, the seventh code segment further includes a twelfth code segment and a thirteenth code segment. The twelfth code segment is provided to remain the matched fragments and the thirteenth code segment is provided to combine all of the extended fragments satisfying the extension condition and the matched fragments into the resulted fragments.

The present invention will become apparent with reference to the below examples. These examples are given by way of illustration only and thus not intended to be any limitation of the present invention.

The first embodiment of the present invention has a specific application to aligning similarity of genome of living organisms, in particular human genome, so as to find biological information therein, such as the fragments of interesting biological sequences.

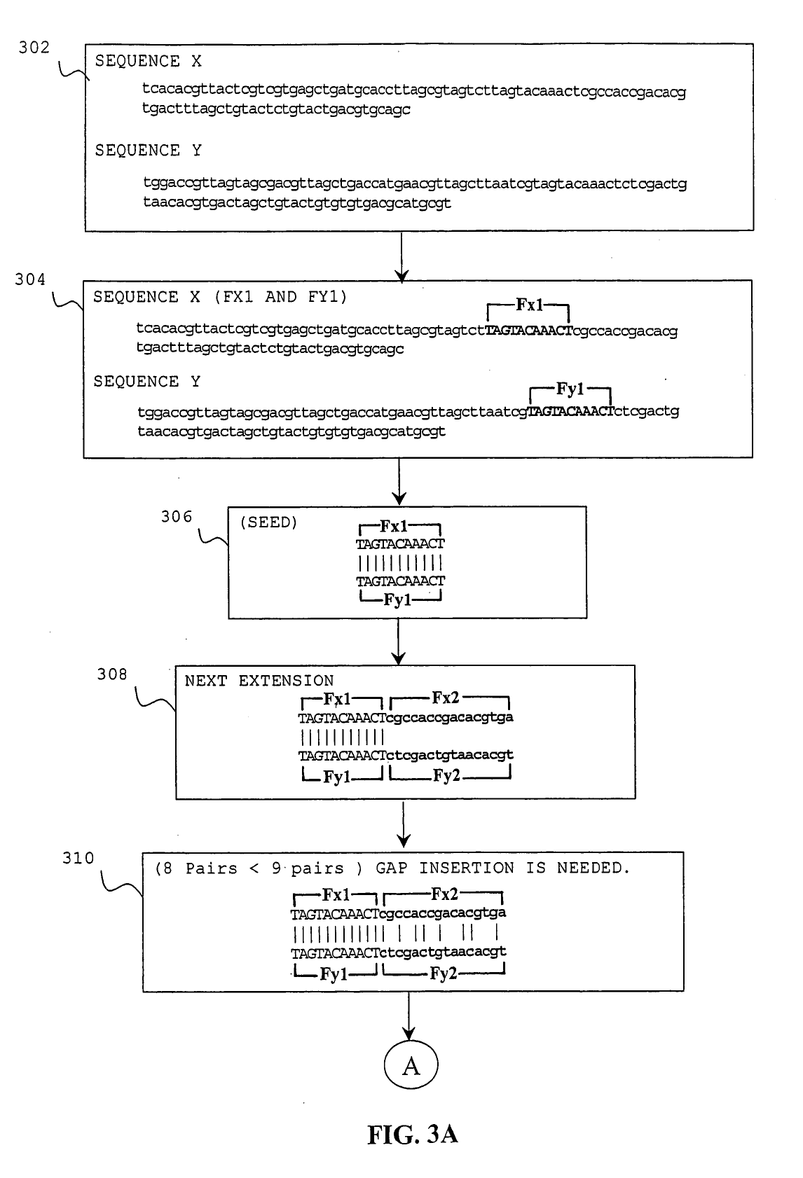

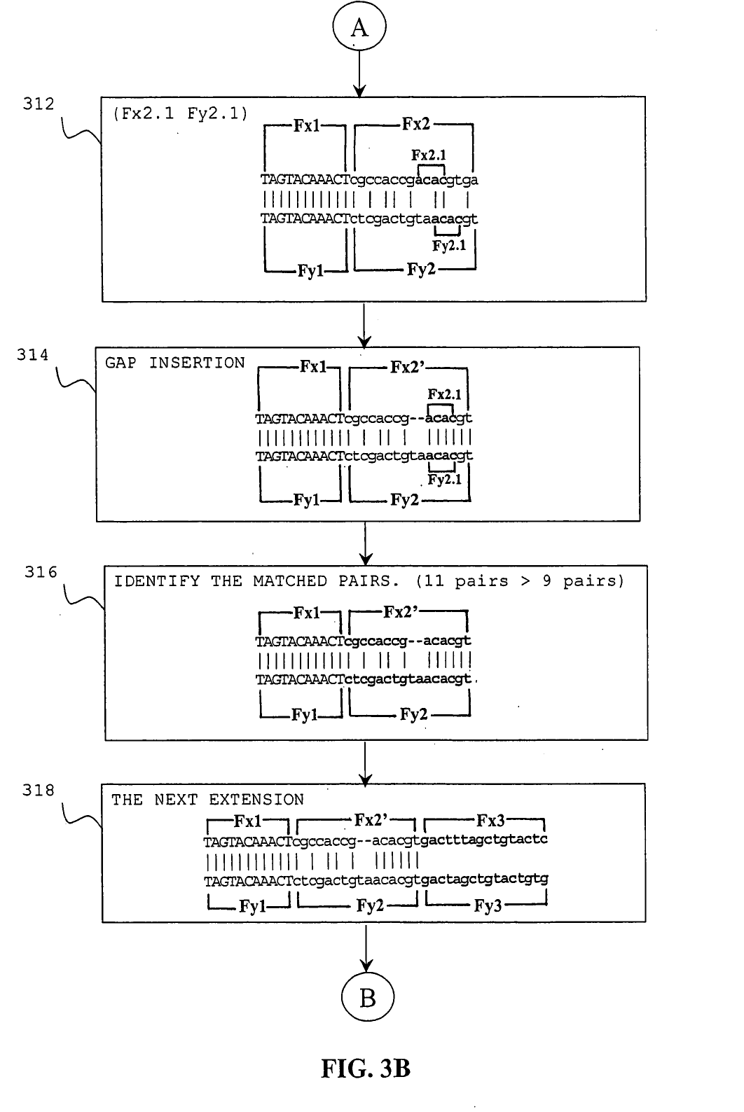

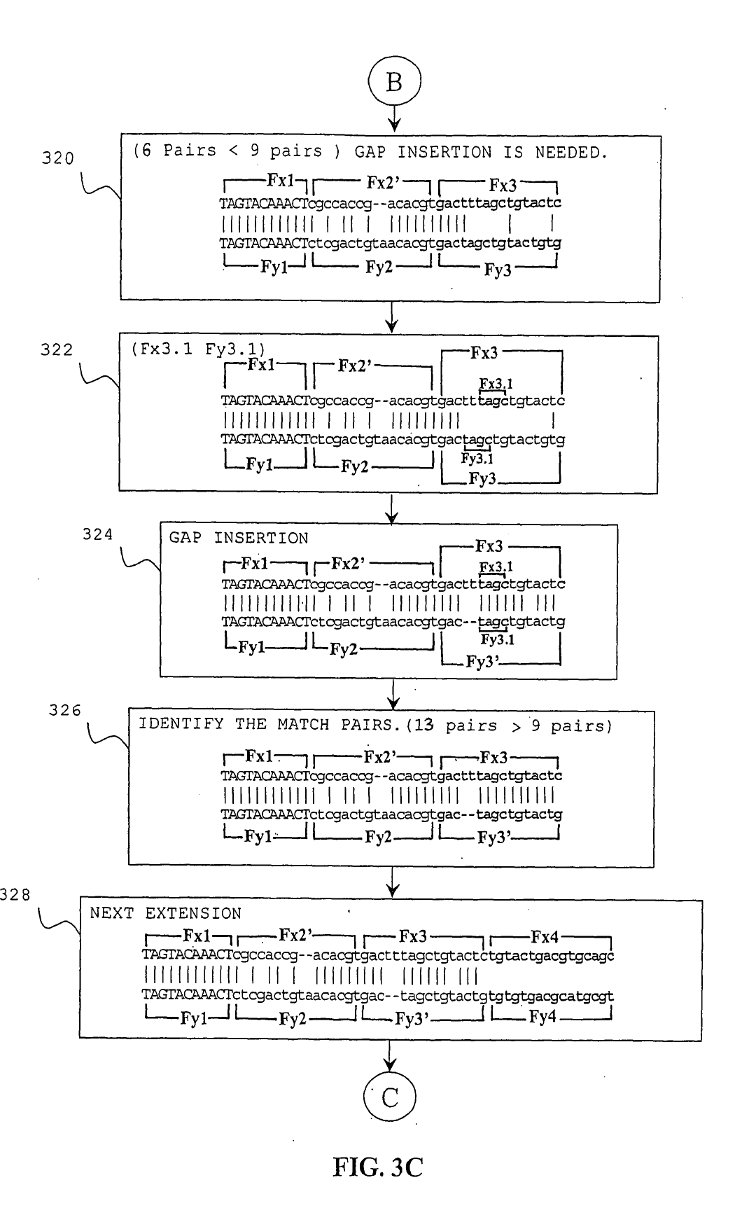

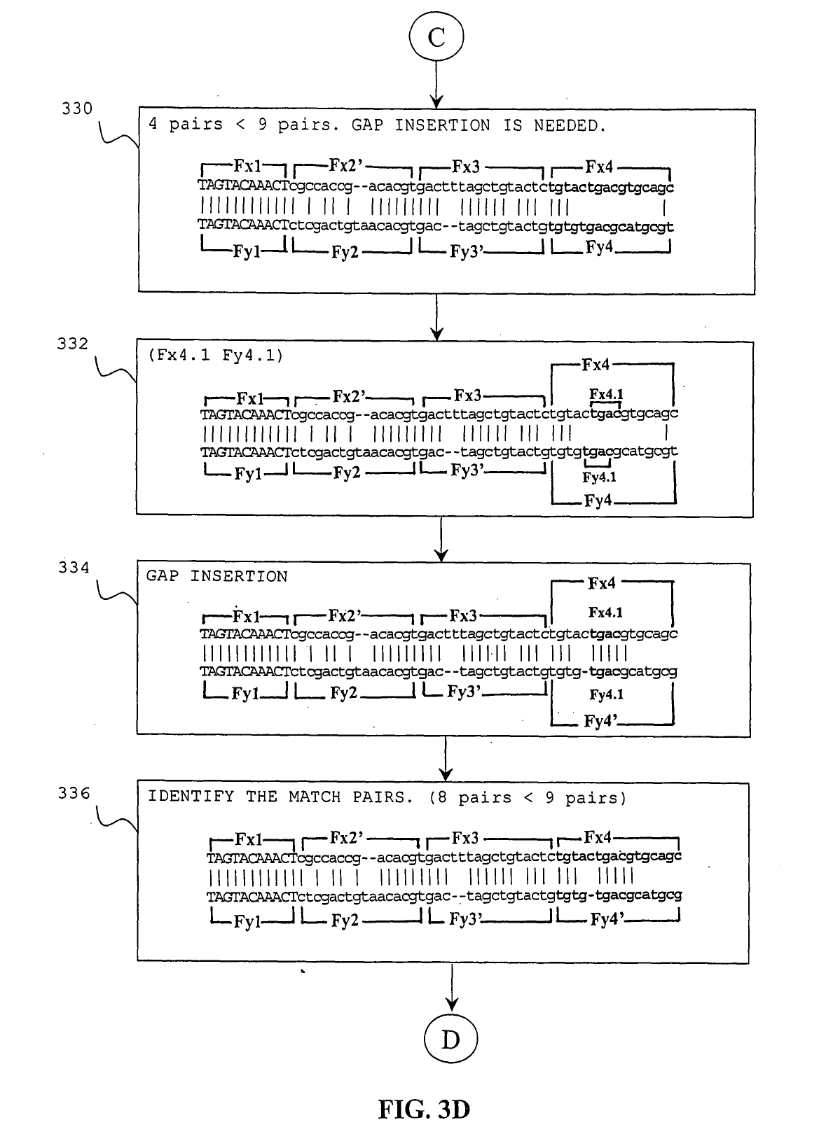

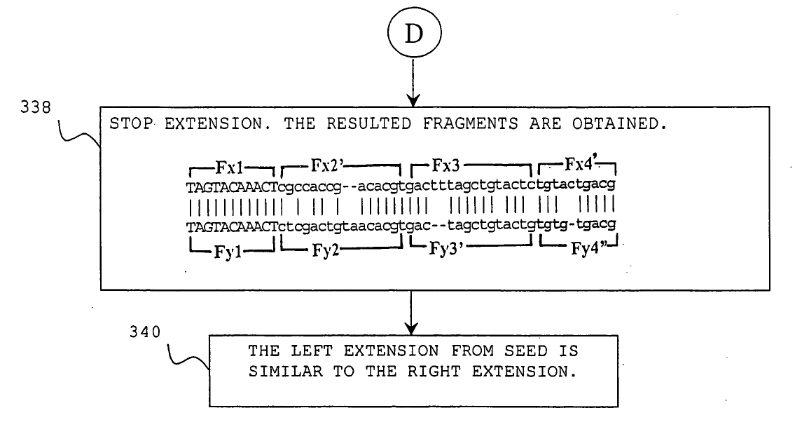

With reference to FIG. 3 (including FIG. 3A, FIG. 3B, FIG. 3C, FIG. 3D and FIG. 3E), there are two DNA sequences to be proceeded, designated as X and Y in block 302. A seed pair, Fx1 and Fy1, is selected by having the same length of 11 identical successive bases in block 304. Fx1 and Fy1 in block 306, as starting points, are going to be extended two fragments in the right direction and the predetermined number of successive bases for extension is 16, designated as Fx2 and Fy2 in block 308. The number of identical bases of Fx2 and Fy2 is detected as 8/16 pairs, which are lower than the user-defined extension condition, 9/16 pairs, in block 310. Thus it is required to match Fx2 and Fy2. The match procedure starts by selecting two sub-fragments, Fx2.1 and Fy2.1, respectively from Fx2 and Fy2 in block 312 by having 4 identical successive bases. Therefore, one can find the same nucleotide bases, “acac”, between Fx2 and Fy2. Two gaps are inserted into the position in front of the first base of Fx2.1 in block 314 since it is closer to the seed pair than Fy2.1. Accordingly, the nucleotide bases of Fx2 are shifted so that an updated fragment is generated, named as Fx2′, in block 316. It is noted that the most right two bases of Fx2, “ga”, are excluded from Fx2′. After gap insertion, the matched number is increased as 11/16 pairs, which are greater than the user-defined extension condition of 9/16 pairs. So the next fragments, Fx3 and Fy3, are extended by 16 successive bases subsequently from Fx2′ and Fy2 in the right direction in block 318.

The number of identical base pairs of Fx3 and Fy3 is detected as 6/16 pairs in block 320, which are lower than the extension condition of 9/16 pairs. The match procedure starts again to attempt to satisfy the extension condition. Two identical sub-fragments, Fx3.1 and Fy3.1, are selected by having 4 identical successive bases as set forth above in block 322. Because Fy3.1 is closer to the seed pair, 2 gaps are inserted into the position in front of the first base of Fy3.1 in block 324 so that Fy3 becomes an updated fragment, named as Fy3′. It is noted that the most right two bases of Fy3, “tg”, are excluded from Fy3′. The matched number of Fy3′ and Fx3 then becomes 13/16 pairs, which are larger than the extension condition of 9/16 pairs in block 326. Therefore next fragments, Fx4 and Fy4, are extended subsequently by 16 successive bases in the right direction in block 328.

The number of identical base pairs of Fx4 and Fy4 is detected as 4/16 pairs, which are lower than the extension condition of 9/16 pairs in block 330. Therefore the match procedure starts again. Two identical sub-fragments, Fx4.1 and Fy4.1, are selected in block 332. One gap is inserted in block 334 so that Fy4 becomes an updated fragment, named as Fy4′. It is noted that the most right base of Fy4, “t”, is excluded from Fy4′. The matched number of Fy4′ and Fx4 turns to be 8/16 pairs, which are still lower than the extension condition of 9/16 pairs in block 336. Finally, the extension process is terminated and the extended fragments which satisfy the extension condition are Fx1+Fx2′+Fx3 and Fy1+Fy2+Fy3′.

Now we need to decide whether Fx4 and Fy4′ should be included in the resulted fragments. Since the preceding bases of Fy4′ and Fx4, “tgtactgacg” and “tgtg-tgacg”, are highly similar (80%), it is suggested to generate another highly similar pair based on Fx4 and Fy4′, named as Fx4′ and Fy4″, by keeping the preceding 10 bases but waiving the rest bases of Fy4′ and Fx4 in block 338. Accordingly, the resulted fragments in the right direction are Fx1+Fx2′+Fx3+Fx4′ and Fy1+Fy2+Fy3′+Fy4″.

After executing the method of the present invention for similarity alignment in the right direction, those skilled in the art will be aware that the left extension from Fx1 and Fy1 can be easily performed with the same principles in block 340 as that of the right extension taught above. To obtain maximum similar fragments, the final resulted fragments may include the resulted fragments in the right direction as well as the resulted fragments in the left direction.

The second embodiment of the present invention is to search for two highly similar biological sequences from a biological sequence database. In this embodiment, the present invention selects one target biological sequence from the database first and each of the rest biological sequences compares with the target biological sequence to determine similarity of the two at one time. For example, if there are 100 biological sequences in a database, the method of the present invention will be executed C₂¹⁰⁰=4950 times to find out similarity of the 100 biological sequences.

Moreover, as a third embodiment of the present invention, the biological sequences in a database are regarded as reference sequences and one new logical sequence excluded from the database can be verified if it is similar to any reference sequence in the database.

To compare the present invention with BLAST 2 sequences (bl2seq), the following is an experiment to evaluate the performances of the two methods. In the experiment, both the present invention and bl2seq are running at their default settings by use of Pentium III CPU (500 MHz) with 1G RAM. There are 4 microbial genome available:

1. cjef

emb|AL111168|AL111168 Campylobacter jejuni complete genome

Length=1641481 bases

2. mgen

gb|L43967|L43967 Mycoplasma genitalium G37 complete genome

Length=580074 bases

3. aful

gb|AE000782|AE000782 Archaeoglobus fulgidus complete genome

Length=2178400 bases

4. bbur

gb|AE000783|AE000783 Genomic sequence of a Lyme disease spirochete, Borrelia burgdorferi

Length=910724 bases

and the experiment results are shown in Table 1. Wherein, “coverage” means the percentage of determined similar bases over all bases of a sequence.

TABLE 1

| | | | | A. fulgidus-B. | |
|---|---|---|---|---|---|
| | | C. jejuni-M. genitalium | | burgdorferi | |
| Microbial genome | | the present | BLAST | the present | BLAST |
| Program | | invention | (bl2seq) | invention | (bl2seq) |
| Speed (seconds) | | 17.3 | 50534 | 24.8 | 14044.9 |
| Alignment | Total aligned bases | 49774 | 12258 | 7760 | 1979 |
| | Matched bases | 36274 | 11183 | 5544 | 1896 |
| | Inserted gaps | 13500 | 1075 | 2216 | 83 |
| Aligned bases | 100-90% | 947 | 5776 | 97 | 1766 |
| in similarity | 89-80% | 3679 | 6472 | 273 | 213 |
| intervals | 79-70% | 30578 | 170 | 4205 | 0 |
| | 69-60% | 14570 | 0 | 3185 | 0 |

| | | |
|---|---|---|
| Coverage (%) | 2.55 | 0.70 |

In terms of speed, the present invention only spend tens seconds, but bl2seq spends ten thousands second. It is explicit that the present invention is hundreds times, even thousands times, faster than Gapped-BLAST.

In terms of amount of alignments, the present invention uses more inserted gaps and obtains more matched bases than bl2seq. That means the present invention can do more gap insertion to obtain more matched bases than BLAST.

In terms of aligned bases in similarity intervals, the present invention is able to align similarity from 60% to 100%. However, bl2seq is only able to align similarity from 70% to 100%. Therefore, the present invention has a larger similarity alignment range.

In terms of coverage, the present invention can obtain 2.55% coverage but bl2seq only obtains 0.70% coverage. Besides, because coverage is proportional to sensitivity. Accordingly, the present invention has higher sensitivity than BLAST.

To conclude, the present invention has better performances in speed, amount of alignment, aligned bases in similarity intervals and coverage.

It should be understood that the preferred embodiment has been presented by way of example only, but not limitation. Thus, the breadth and scope of a preferred embodiment should not be limited by any of the aforementioned exemplary embodiments, but should be defined only in accordance with the following claims and their equivalents.

1. A method for aligning similarity of two biological sequences, the biological sequences consisting of bases, the method comprising the steps of:
(a) selecting a seed pair of the two biological sequences;
(b) respectively extending two fragments adjacent to the a seed pair by a predetermined number of successive bases;
(c) determining if the extended fragments satisfy an extension condition, if yes, going to step (d), if no, going to step (e);
(d) extending respectively two fragments adjacent to the extended fragments by the predetermined number of successive bases and returning to step (c);
(e) respectively selecting two identical sub-fragments from the extended fragments unsatisfying the extension condition;
(f) determining either one of the sub-fragments closer to the a seed pair;
(g) matching the extended fragments by inserting at least one gap in front of the one of the sub-fragments determined in step (f);
(h) determining if the matched fragments satisfy the extension condition, if yes, going to step (i), if no, going to step (j);
(i) respectively extending two fragments adjacent to the matched fragments by the predetermined number of successive bases and returning to step (c); and
(j) stopping extension and obtaining resulted fragments.

2. The method of claim 1, wherein the predetermined number of successive bases is from 4 to 400.

3. The method of claim 1, wherein the extension condition comprises having 40%˜100% similarity of fragments.

4. The method of claim 1, wherein a base number of the two identical sub-fragments is at least 2.

5. The method of claim 4, wherein a base number of the two identical sub-fragments is from 3 to 400.

6. The method of claim 5, wherein a base number of the two identical sub-fragments is from 3 to 50.

7. The method of claim 1, wherein step (j) further comprises:
(k) intercepting preceding substantially identical bases of the matched fragments; and
(l) combining all of the extended fragments satisfying the extension condition and the intercepted bases into the resulted fragments.

8. The method of claim 1, wherein step (j) further comprises:
(m) waiving the matched fragments; and
(n) combining all of the extended fragments satisfying the extension condition into the resulted fragments.

9. The method of claim 1, wherein step (j) further comprises:
(o) remaining the matched fragments; and
(p) combining all of the extended fragments satisfying the extension condition and the matched fragments into the resulted fragments.

10. A computer program product for aligning similarity of two biological sequences, the biological sequences consisting of bases, the computer program product comprising:
a computer readable storage medium having code segments embodied therein, the code segments comprising:
a first code segment configured to select a seed pair of the two biological sequences;
a second code segment configured to respectively extend two fragments by a predetermined number of successive bases;
a third code segment configured to determine whether the extended fragments satisfy an extension condition;
a fourth code segment configured to respectively select two identical sub-fragments from the extended fragments;
a fifth code segment configured to determine either one of the sub-fragments closer to the a seed pair;
a sixth code segment configured to match the extended fragments by inserting at least one gap in front of the one of the sub-fragments; and
a seventh code segment configured to obtain resulted fragments.

11. The computer program product of claim 10, wherein the predetermined number of successive bases is from 4 to 400.

12. The computer program product of claim 10, wherein the extension condition comprises having 40%˜100% similarity of fragments.

13. The computer program product of claim 10, wherein a base number of the two identical sub-fragments is at least 2.

14. The computer program product of claim 13, wherein a base number of the two identical sub-fragments is from 3 to 400.

15. The computer program product of claim 14, wherein a base number of the two identical sub-fragments is from 3 to 50.

16. The computer program product of claim 10, wherein the seventh code segment further comprises:
an eighth code segment configured to intercept preceding substantially identical bases of the matched fragments; and
a ninth code segment configured to combine all of the extended fragments satisfying the extension condition and the intercepted bases into the resulted fragments.

17. The computer program product of claim 10, wherein the seventh code segment further comprises:
a tenth code segment configured to waive the matched fragments; and
an eleventh code segment configured to combine all of the extended fragments satisfying the extension condition into the resulted fragments.

18. The computer program product of claim 10, wherein the seventh code segment further comprises:
a twelfth code segment configured to remain the matched fragments; and
a thirteenth code segment configured to combine all of the extended fragments satisfying the extension condition and the matched fragments into the resulted fragments.


SEQUENCE LISTING (2 sequences)

SEQ ID NO: 1 - DNA, 95 bp, Artificial
  Synthesized sequence
tcacacgttactcgtcgtgatgcaccttagcgtagtcttagtacaaactcgccaccgacacgtgactttagctgtactctgtactgacgtgcagc

SEQ ID NO: 2 - DNA, 105 bp, Artificial
  Synthesized sequence
tggaccgttagtagcgacgttagctgaccatgaacgttagcttaatcgtagtacaaactctcgactgtaacacgtgactagctgtactgtgtgtgacgcatgcgt